(19) United States
(12) Patent Application Publication
(10) Pub. No.: US 2025/0388955 A1
(43) Pub. Date: Dec. 25, 2025

(54) METHYLATION DETECTION WITH A NON-NATURAL/UNNATURAL BASE

(71) Applicant: ILLUMINA, INC., San Diego, CA (US)

(72) Inventors: Jeffrey FISHER, San Diego, CA (US); Yin Nah TEO, Singapore (SG); Paul VAN HUMMELEN, San Diego, CA (US); Sergio PEISAJOVICH, San Diego, CA (US); Molly HE, San Diego, CA (US)

(21) Appl. No.: 18/879,632

(22) PCT Filed: Jun. 27, 2023
(86) PCT No.: PCT/US2023/069202

§ 371 Date: Dec. 27, 2024

Related U.S. Application Data
(60) Provisional application No. 63/357,147, filed on Jun. 30, 2022.

(51) Int. Cl. C12Q 1/6858

(52) U.S. Cl. CPC C12Q 1/6858; C12Q 2521/301; C12Q 2521/531; C12Q 2525/119

(57) ABSTRACT

The present application provides a method for detecting methylated cytosine in a double stranded target polynucleotide. A double stranded target polynucleotide is treated with an enzyme having glycosylase activity that selectively removes methylated cytosine so as to create an abasic site. The phosphate backbone of the target polynucleotide is broken at the abasic site with an AP lyase or AP endonuclease. Depending on the nature of the backbone cleavage reaction, it may be necessary to provide a 3′ hydroxyl and/or a S5′ triphosphate group. The abasic site is then repaired by inserting a non-natural base into the abasic site to generate repaired target polynucleotide. The repaired target polynucleotide then contains the non-natural/unnatural base so as to identify positions in the repaired target polynucleotide that contained methylated cytosine in the target polynucleotide.

### TECHNICAL FIELD

The present invention relates to a method and kits for the detection of a methylated cytosine, 5-methylcytosine (5mC) and/or 5-hydroxymethylcytosine (5hmC), by replacement with a non-natural/unnatural base pair.

### BACKGROUND

Epigenetic modifications, such as the methylation of the C5 position of cytosine, typically in a CpG dinucleotide, is an essential process in normal development and is involved in several key physiological processes such as regulation of gene expression, X-chromosome inactivation, imprinting, silencing of germ-line-specific genes and repetitive elements, and maintenance of chromosomal stability. These modifications are also involved in the onset and progression of human diseases such as imprinting disorders and cancer. In addition, cellular methylation patterns can provide information on the cell of origin, stage of cell/tissue differentiation, and can potentially discriminate stages in cancer progression.

In contrast, recurrent methylation patterns across different cancers may aid the development of diagnostic and prognostic biomarkers and improve patient stratification and the discovery of novel drug targets for therapy. A comprehensive understanding of the role of genome-wide DNA methylation patterns, the methylome, requires quantitative determination of the methylation states of all the CpG sites in a genome. The most common method for DNA methylation analysis is genome sequencing of bisulfite converted DNA.

The method utilizing bisulfate conversion takes advantage of the increased sensitivity of cytosine, relative to 5-methylcytosine (5-meC) and 5-hydroxymethylcytosine (5hmC), to bisulfite deamination under acidic conditions. This deamination results in a conversion of non-methylated cytosine to uracil, which is then read by polymerases as a thymine during sequencing reactions. Comparison of a bisulfite treated target nucleic acid to a non-bisulfite treated nucleic acid allows for those sites that read as cytosine in the non-bisulfite treated sample, but read as thymine in the bisulfite treated sample, to be inferred as having been non-methylated cytosine. Those cytosine bases that continued to be read as cytosine in the bisulfite treated target are inferred to have been methylated.

However, there are a number of limitations to the bisulfite treatment method. First, the bisulfite treatment protocol is chemically harsh, and results in large amounts of DNA loss, which necessitates significantly more input genomic material. Second, prolonged bisulfite treatment causes the sample to degrade in a way which enriches the small amount of remaining material for methylated reads. However, if the bisulfite conversion does not run to completion, unmethylated cytosines will be indistinguishable from methylated cytosines, and thus introduce false positive methylation calls. Third, to avoid non-conversion errors and to estimate the bisulfite conversion rate, the same reactions and times need to be applied to a known control sequence. For example, a known sequence with known levels of methylation is used (see, e.g. https://support.illumina.com/bulletins/2017/02/how-much-phix-spike-in-is-recommended-when-sequencing-low-divers.html, which is incorporated by reference in its entirety). This requires more sequencing reads. In addition, controls might not have the same conversion properties as the sample to be analyzed. Fourth, in recent years, methylation sites have been found in non-CPG sites. These sites are not well detected in bisulfite sequencing. Only 5-MeC in CpG sites can be reliably detected. Fifth, bisulfite sequencing relies on the complete conversion of unmodified cytosine to uracil. Unmodified cytosine accounts for approximately 95% of the total cytosine in the human genome. Converting all these positions to uracil severely reduces sequence complexity, leading to poor sequencing quality, low mapping rates, uneven genome coverage, and increased sequencing cost. Finally, the methylation state of bisulfite treated DNA must be inferred by comparison to an unmodified reference sequence. Thus, a correct alignment is very important.

Bisulfite sequencing methods, including but not limited to, Tet-assisted bisulfite sequencing and oxidative bisulfite sequencing, can also be challenging if the aligned sequences do not exactly match the reference.

Also, cytosine methylation is not symmetrical, thus the two strands of DNA in the target sequence may need to be considered separately. In addition, a single site can have different methylation state in different cells. Four DNA strands can arise through bisulfite treatment and subsequent PCR since the top and bottom strands are methylated differently. Bisulfite sequence mapping therefore may require up to four different strand alignments to be analyzed for each sequence. This increases the complexity of sequence alignments and standard sequence alignment software cannot be used.

### SUMMARY

Disclosed herein are methods of detecting methylated cytosine in a target DNA. Such methods comprise treating the target DNA with an enzyme having DNA glycosylase activity that selectively removes methylated cytosine so as to create an abasic site; breaking the phosphate backbone of the target DNA at the abasic site with a DNA AP lyase or AP endonuclease; repairing the abasic site by inserting a non-natural base into the abasic site to generate repaired target DNA; and sequencing the repaired target DNA so as to identify positions in the repaired target DNA that contain the non-natural base thereby detecting methylated cytosine in the target DNA.

In particular methods, the non-natural base may be a low-fidelity base that is capable of forming non-specific base pairs. In other methods, the non-natural base may be a base other than A, T, C, G, or U that forms a specific base pair with another non-natural base.

Further disclosed herein are kits for carrying out the methods described herein. Such kits may contain an enzyme having DNA glycosylase activity that selectively removes methylated cytosine so as to create an abasic site; a DNA AP lyase or AP endonuclease which is capable of breaking the phosphate backbone of the target DNA at the abasic site; and at least one a non-natural base capable of repairing the abasic site by inserting into the abasic site to generate repaired target DNA.

### BRIEF DESCRIPTION OF THE DRAWINGS

FIG. 1 is a conceptual view schematically showing a method for methylation detection with a non-natural/unnatural base pair by replacement of methylated cytosine with a non-natural/unnatural base.

FIG. 2 is a conceptual view schematically showing a method for methylation detection by replacement of methylated cytosine with deoxyinosine.

FIG. 3 depicts the structures of three exemplary non-natural/unnatural base pairs suitable for use in the described methods.

FIG. 4 depicts possible methods for the detection of a fifth type of base via fluorescence.

FIG. 5 depicts a scheme for the identification of the site of a methylated cytosine by the identification of multiple different nucleotides being incorporated at a particular location.

### DETAILED DESCRIPTION OF THE INVENTION

The present invention provides a new method for detecting methylated cytosines in nucleic acids, such as genomic DNA. In the present invention a methylated cytosine is detected by replacement with a non-natural/unnatural base pair.

In an exemplary embodiment, the present invention provides a method for detecting methylated cytosine in a double stranded target polynucleotide. A double stranded target polynucleotide is treated with an enzyme having glycosylase activity that selectively removes methylated cytosine so as to create an abasic site. The phosphate backbone of the target polynucleotide is broken at the abasic site with an AP lyase or AP endonuclease. Depending on the nature of the backbone cleavage reaction, it may be necessary to provide a 3′ hydroxyl and/or a 5′ triphosphate group. The abasic site is then repaired by inserting a non-natural base into the abasic site to generate repaired target polynucleotide. The repaired target polynucleotide then contains the non-natural/unnatural base so as to identify positions in the repaired target polynucleotide that contained methylated cytosine in the target polynucleotide.

The invention includes, but is not limited to, selectively excising 5-meC and/or 5-hmeC from a target nucleic acid, inserting a non-natural/unnatural base in the apurinic/apyrimidinic site (abasic/AP site) to create a repaired target nucleic acid, which can then be read as positions formerly containing a 5-mwC and/or 5-hmeC in the repaired target nucleic acid.

As used herein, “non-natural base” and/or “unnatural base” is a nucleotide that can be incorporating into a nucleic acid that is not A, T, G, C, or U. Examples of such non-natural/unnatural bases include, but are not limited to dDs, dPx, dP, dZ, dNam, D5SICS, deoxyinosine, and 5-nitroindole. As used herein, “non-natural base pairs” and/or “unnatural base pairs” are base pairs in a double stranded nucleic acid that include on or more non-natural/unnatural bases.

The present invention allows for the omission of bisulfite conversion completely.

### Base Excision

Disclosed herein are methods of detecting methylated cytosine in a target DNA. Such methods comprise treating double stranded target DNA with an enzyme having DNA glycosylase activity that selectively removes methylated cytosine so as to create an abasic site; breaking the phosphate backbone of the target DNA at the abasic site with a DNA AP lyase or AP endonuclease; repairing the abasic site by inserting a non-natural base into the abasic site to generate repaired target DNA; and sequencing the repaired target DNA so as to identify positions in the repaired target DNA that contain the non-natural base thereby detecting methylated cytosine in the target DNA.

In an exemplary embodiment, the base excision enzyme is glycosylase which will selectively remove a methylated cytosine base. In particular embodiments, the glycosylase will have EC 3.2.2.-activity. Examples of proteins having the required glycosylase activity include, but are not limited to, transcriptional activator DEMETER, DNA glycosylase/AP lyase ROS1, DEMETER-like protein 2 (DML2), DEMETER-like protein 3 (DML3) (and related proteins from species other than Arabidopsis, for example, E. coli Nth, and Homo sapiens MutY and Ogg1. Another exemplary glycosylase includes, but is not limited to, methyl-CpG-binding domain protein 4 (MBD4). Proteins in other organisms that are homologous, analogous and/or paralogous may also be used, for example, non-Arabidopsis proteins include, but are not limited to, APE1/Ref-1/APEX1. All four of DEMETER, ROS1, DML2, and DML3, are bifunctional enzymes, possessing both glycosylase (base excision) and AP lyase activity.

### Backbone Cleavage and Preparation

Once the abasic site is created, the backbone of the nucleic acid is broken at the abasic site. In embodiments, the breaking of the nucleic acid backbone is catalyzed by an enzyme having AP lyase and/or AP endonuclease activity. The AP endonuclease may be a Class I, Class II, or Class III endonuclease. In particular embodiments, the AP lyase and/or AP endonuclease activity may have EC 4.2.99.18 activity.

In an exemplary embodiment, a glycosylase may be monofunctional and comprise glycosylase activity without AP lyase activity, in a second exemplary embodiment a glycosylase may be bifunctional and comprise both glycosylase activity and AP lyase activity. For example, ROS1. In another exemplary embodiment, the glycosylase comprises apurinic and/or apyrimidinic site endonuclease activity. In another exemplary embodiment an endonuclease may be utilized to introduce a break in the phosphodiester bond, creating a single-strand break, and or to prepare the break for incorporation of a nucleotide.

β-Elimination of an AP site by a glycosylase-lyase yields a 3′ α,β-unsaturated aldehyde adjacent to a 5′ phosphate, which differs from an AP endonuclease cleavage product. Some glycosylase-lyases can further perform δ-elimination, which converts the 3′ aldehyde to a 3′ phosphate. A 3′ α,β-unsaturated aldehyde is not compatible with direct insertion of a non-natural/un-natural triphosphate base, therefore conversion of the a 3′ α,β-unsaturated aldehyde to a 3′ hydroxyl is required prior to ligation of the non-natural/unnatural nucleotide into the target nucleic acid.

An endonuclease, such as endonuclease IV, and/or a 3′ phosphatase may be used to prepare the abasic cleavage site for base incorporation, depending on the nature of the nick or single strand break. In an exemplary embodiment, an endonuclease comprising EC 3.1.21.2 and/or EC 3.1.21.9 activity is utilized. For example, endonuclease II and/or IV. In embodiments, the 3′ phosphatase may be a 3′ phosphatase comprising EC 3.1.3.32 activity.

Repair of the Abasic Site with a Non-Natural/Unnatural Base

The double stranded nucleic acid may then be incubated with a non-natural/unnatural base, so that a polymerase will incorporate this non-natural/unnatural base into the abasic site. A ligase may then be used to close the backbone at the site of the incorporated base to thus form a repaired nucleic acid comprising a non-natural/unnatural base at the site of a methylated cytosine.

In an exemplary embodiment, a polymerase comprising EC 2.7.7.6, EC 2.7.7.7, and/or EC 2.7.7.49 activity. The polymerase may be a DNA-directed RNA polymerase, a DNA-directed DNA polymerase and/or an RNA-directed DNA polymerase. Exemplary polymerases include, but are not limited to, TaqDNA polymerase (from Thermis aquaticus), PfuDNA polymerase (from Pyrococcus furiosus), BstDNA Polymerase I (from Bacillus stearothermophilus), Vent polymerase (from Pyrococcus), Deep Vent polymerase (from Pyrococcus) and UlTma DNA polymerase (from Thermotoga maritima), see Ishino S, Ishino Y. DNA polymerases as useful reagents for biotechnology—the history of developmental research in the field. Front Microbiol. 2014; 5:465. Published 2014 Aug. 29. doi:10.3389/fmicb.2014.00465, which is incorporated by reference in its entirety.

In an exemplary embodiment, a ligase comprising EC 6.5.1 EC 6.5.1.1 and/or EC 6.5.1.2, EC 6.5.1.6 and/or EC 6.5.1.7 activity is utilized to seal a single-strand break in the repaired target nucleic acid. For example, joining a 3′-hydroxyl and 5′-phosphate termini, forming a phosphodiester to seal a single-strand break.

Repair of the Abasic Site with a Low Fidelity Non-Natural/Unnatural Base

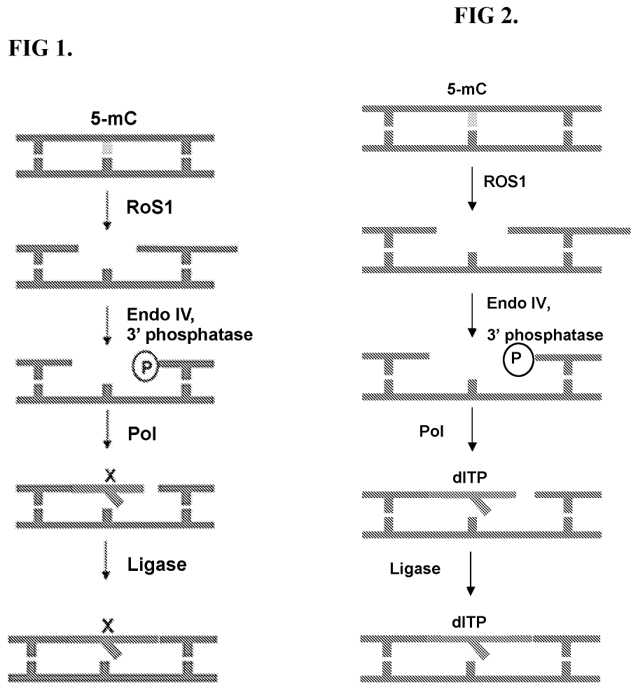

In an exemplary embodiment, the non-natural/unnatural base pairs with a multitude of the natural bases with low fidelity for any particular natural base. One non-limiting example of the process leading to the incorporation of a low fidelity non-natural/unnatural base is provided in FIG. 1. Therein, 5-mC is removed by ROS1. Endonuclease IV and a 3′ phosphatase are then utilized to prepare the abasic site. A polymerase is then used add a low fidelity non-natural/unnatural base into the gap, in this case deoxyinosine. A ligase is then used to seal the backbone.

The location of the non-natural/unnatural base is then identified by a fidelity error rate above the background error rate and/or with a statistically significant rate of perceived error above background.

In one exemplary embodiment, the non-natural/unnatural base comprises deoxyinosine or 5-Nitroindole nucleosides as a universal base in a non-natural/unnatural nucleotide. Loakes D, Brown DM. 5-Nitroindole as a universal base analogue. Nucleic Acids Res. 1994;22(20):4039-4043. doi:10.1093/nar/22.20.4039, the entirety of which is incorporated by reference. In another exemplary embodiment, the non-natural/unnatural base comprises 3-methyl 7-propynyl isocarbostyril (PIM), 3-methyl isocarbostyril (MICS), or 5-methyl isocarbostyril (5MICS) nucleosides as a universal base in a non-natural/unnatural nucleotide. Berger M, Wu Y, Ogawa A K, McMinn D L, Schultz P G, Romesberg F E. Universal bases for hybridization, replication and chain termination. Nucleic Acids Res. 2000;28(15):2911-2914. doi:10.1093/nar/28.15.2911, the entirety of which is incorporated by reference.

Repair of the Abasic Site with a High-Fidelity Non-Natural/Unnatural Base

In addition to low fidelity non-natural/unnatural bases, the non-natural/unnatural base may pair with high fidelity to a second non-natural/unnatural base. One non-limiting example of the process leading to the incorporation of a low fidelity non-natural/unnatural base is provided in FIG. 2. Therein, 5-mC is removed by ROS1. Endonuclease IV and a 3′ phosphatase are then utilized to prepare the abasic site. A polymerase is then used add a high-fidelity non-natural/unnatural base into the gap. A ligase is then used to seal the backbone.

The research group of Professor Ichiro Hirao developed non-natural/unnatural base pairs, such as the DS-PX pair (U.S. Pat. Nos. 7,667,031 and 8,030,478, the entirety of both are hereby incorporated by reference) (FIG. 2b). Previous work showed that DNA fragments containing Ds and Px are amplified 1028-fold after 100 cycles of PCR and more than 97% of the DS-PX pairs were maintained in the amplified DNA. This suggests that DNA molecules containing the Ds and Px can be amplified by Polymerase Chain Reaction (PCR) with high efficiency and fidelity.

In recent years, the Romesberg group has also developed a multitude of non-natural/unnatural base pairs, including, but not limited to, a hydrophobic NaM-5SICS (3-methoxy-2-naphthyl (NaM) paired with 6-methylisoquinoline-1-thione-2-yl (d5SICS), which pairs with an artificial nucleobase containing a group instead of a natural base (dNaM)) base pair (FIG. 2d). This non-natural/unnatural base pair is an example of a non-natural/unnatural base pair that can be amplified with selectivity of between approximately 99.6 to 100% using KlenTaq polymerase. It has also been shown to be replicated in vivo, with fidelity of about 99.4%. This is comparable to the intrinsic error rate of some polymerases with natural DNA.

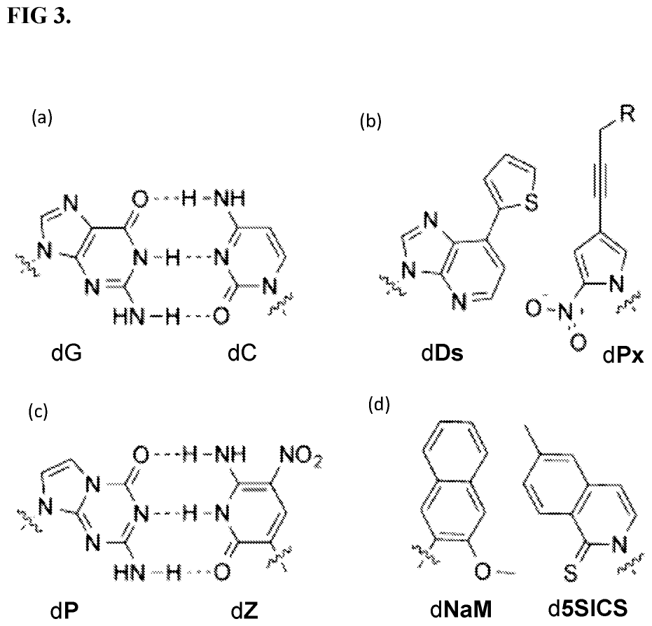

Another base pair with more than 99% selectivity is the P-Z base pair (2-aminoimidazo[1,2-a]1,3,5-triazin-4(8H)-one (P) and 6-amino-5-nitro2(1H)-pyridone (Z)) developed by the Benner group (FIG. 3c). See U.S. Pat. No. 7,794,984 and US Patent Publication No. 2020/0040027, the entirety of both is hereby incorporated by reference. The selectivity and misincorporation rate of the P-Z base pair is at least 99.8% per replication and 0.2% per base per replication. These exemplary non-natural/unnatural base pairs have been shown to function as a third base pair in replication, transcription and/or translation, demonstrating their high fidelity for their complementary partner.

In one exemplary embodiment, the non-natural/unnatural base pair comprises 7-(2-thienyl)-imidazo[4,5-b]pyridine (Ds) and pyrrole-2-carbaldehyde (Pa), which pair by specific hydrophobic shape complementation. The Ds-Pa pair functions as a template base pair when used with exonuclease-proficient (exo+) DNA polymerases, such as, but not limited to, the Klenow fragment, Dpo4 and Vent DNA polymerases, as well as the T7 RNA polymerase. In another exemplary embodiment the non-natural/unnatural base pair comprises Ds and 4-[3-(6-aminohexanamido)-1-propynyl]-2-nitropyrrole (Px).

In another exemplary embodiment, the non-natural/unnatural base pair comprises 2-amino-6-(2-thienyl) purine(S) and 2-oxopyridine (Y). In another exemplary embodiment the non-natural/unnatural base pair comprises S and pyrrole-2-carbaldehyde (Pa).

In further embodiments, the non-natural/unnatural base pair may comprise one or more of isoguanine (isoG, 6-amino-2-ketopurine); isocytosine (isoC, 2-amino-4-ketopyrimidine); xDNA and yDNA where the bases are size expanded DNA with their pairing edges shifted by a benzo group e.g. dxT: 1′-b-[8-(6-methylquinazoline-2, 4-dione)]-2′-D-deoxyribofuranosyl and dxA: 3-[2′-Deoxy-D-ribofuranosyl]-8-aminoimidazo[4,5-g]quinazoline.

### Sequencing of Repaired Nucleic Acid Comprising a Non-Natural/Unnatural Base

This non-natural/unnatural base can then be identified by sequencing with its complementary base(s), for example, using a sequencing-by-synthesis reaction.

Non-natural/unnatural base pairs can be amplified with any polymerase capable of incorporating the non-natural/unnatural base(s). For example, Deep Vent (exo+) and AccuPrime (exo+) polymerases. AccuPrime (exo+) polymerase has been shown to incorporate non-natural/unnatural bases in a sequence context, with >99.7% fidelity. Kimoto M, Yamashige R, Yokoyama S, Hirao I. PCR amplification and transcription for site-specific labeling of large RNA molecules by a two-unnatural-base-pair system. J Nucleic Acids. 2012;2012:230943. doi:10.1155/2012/230943, hereby incorporated by reference in its entirety.

The methods described herein can be used in conjunction with a variety of nucleic acid sequencing techniques. Particularly applicable techniques are those wherein nucleic acids are attached at fixed locations in an array such that their relative positions do not change and wherein the array is repeatedly imaged. Embodiments in which images are obtained in different color channels, for example, coinciding with different labels used to distinguish one nucleotide base type from another are particularly applicable.

In some embodiments, the process to determine the nucleotide sequence of a target nucleic acid can be an automated process. An exemplary embodiment includes sequencing-by-synthesis (“SBS”) techniques. Where sequencing by synthesis is used in combination with a high-fidelity non-natural/unnatural base pair, a polymerase that is able to incorporate the a high-fidelity non-natural/unnatural bases is used. Exemplary polymerases have greater than 50%, 60%, 70%, 75%, 80%, 85%, 90%, 91%, 92%, 93%, 94%, 95%, 96%, 97%, 98% and/or 99% fidelity during incorporation of the non-natural/unnatural bases during amplification of the repaired target polynucleotide.

Sequencing techniques can utilize nucleotide monomers that have one or more label moiety(ies) or those that lack a label moiety. Accordingly, incorporation events can be detected based on a characteristic of the label, such as fluorescence of the label; a characteristic of the nucleotide monomer such as molecular weight or charge; a byproduct of incorporation of the nucleotide, such as release of pyrophosphate; or the like. In embodiments, where two or more different nucleotides are present in a sequencing reagent, the different nucleotides can be distinguishable from each other, or alternatively, the two or more different labels can be the indistinguishable under the detection techniques being used. For example, the different nucleotides present in a sequencing reagent can have different labels and they can be distinguished using appropriate optics as exemplified by the sequencing methods developed by Solexa (now Illumina, Inc.).

Other exemplary embodiments include pyrosequencing techniques. Pyrosequencing detects the release of inorganic pyrophosphate (PPi) as particular nucleotides are incorporated into the nascent strand (Ronaghi, M., Karamohamed, S., Pettersson, B., Uhlen, M. and Nyren, P. (1996) “Real-time DNA sequencing using detection of pyrophosphate release.” Analytical Biochemistry 242(1), 84-9; Ronaghi, M. (2001) “Pyrosequencing sheds light on DNA sequencing.” Genome Res. 11(1), 3-11; Ronaghi, M., Uhlen, M. and Nyren, P. (1998) “A sequencing method based on real-time pyrophosphate.” Science 281(5375), 363; U.S. Pat. Nos. 6,210,891; 6,258,568 and 6,274,320, the disclosures of which are incorporated herein by reference in their entireties). In pyrosequencing, released PPi can be detected by being immediately converted to adenosine triphosphate (ATP) by ATP sulfurylase, and the level of ATP generated is detected via luciferase-produced photons. The nucleic acids to be sequenced can be attached to features in an array and the array can be imaged to capture the chemiluminescent signals that are produced due to incorporation of a nucleotides at the features of the array. An image can be obtained after the array is treated with a particular nucleotide type (e.g. A, T, C, G or a non-natural/unnatural base (X)). Images obtained after addition of each nucleotide type will differ with regard to which features in the array are detected. These differences in the image reflect the different sequence content of the features on the array. However, the relative locations of each feature will remain unchanged in the images. The images can be stored, processed and analyzed.

In another exemplary type of cycle sequencing is accomplished by stepwise addition of reversible terminator nucleotides containing, for example, a cleavable or photo bleachable dye label as described, for example, in WO 04/018497 and U.S. Pat. No. 7,057,026, the disclosures of which are incorporated herein by reference. This approach is being commercialized by Solexa (now Illumina Inc.), and is also described in WO 91/06678 and WO 07/123,744, each of which is incorporated herein by reference. The availability of fluorescently-labeled terminators in which both the termination can be reversed and the fluorescent label cleaved facilitates efficient cyclic reversible termination (CRT) sequencing. Polymerases can also be co-engineered to efficiently incorporate and extend from these modified nucleotides.

Preferably in reversible terminator-based sequencing embodiments, the labels do not substantially inhibit extension under SBS reaction conditions. However, the detection labels can be removable, for example, by cleavage or degradation. Images can be captured following incorporation of labels into arrayed nucleic acid features. In particular embodiments, each cycle involves simultaneous delivery of four different nucleotide types to the array and each nucleotide type has a spectrally distinct label. Four images can then be obtained, each using a detection channel that is selective for one of the four different labels. Alternatively, different nucleotide types can be added sequentially and an image of the array can be obtained between each addition step. In such embodiments each image will show nucleic acid features that have incorporated nucleotides of a particular type. Different features will be present or absent in the different images due the different sequence content of each feature. However, the relative position of the features will remain unchanged in the images. Images obtained from such reversible terminator-SBS methods can be stored, processed and analyzed as known in the art. Following the image capture step, labels can be removed and reversible terminator moieties can be removed for subsequent cycles of nucleotide addition and detection. Removal of the labels after they have been detected in a particular cycle and prior to a subsequent cycle can provide the advantage of reducing background signal and crosstalk between cycles.

In particular embodiments some or all of the nucleotide monomers can include reversible terminators. In such embodiments, reversible terminators/cleavable fluorophore can include fluorophore linked to the ribose moiety via a 3′ ester linkage (Metzker, Genome Res. 15:1767-1776 (2005), which is incorporated herein by reference). Other approaches have separated the terminator chemistry from the cleavage of the fluorescence label (Ruparel et al., Proc Natl Acad Sci USA 102:5932-7 (2005), which is incorporated herein by reference in its entirety). Ruparel et al described the development of reversible terminators that used a small 3′ allyl group to block extension, but could easily be deblocked by a short treatment with a palladium catalyst. The fluorophore was attached to the base via a photocleavable linker that could easily be cleaved by a 30 second exposure to long wavelength UV light. Thus, either disulfide reduction or photocleavage can be used as a cleavable linker. Another approach to reversible termination is the use of natural termination that ensues after placement of a bulky dye on a dNTP. The presence of a charged bulky dye on the dNTP can act as an effective terminator through steric and/or electrostatic hindrance. The presence of one incorporation event prevents further incorporations unless the dye is removed. Cleavage of the dye removes the fluor and effectively reverses the termination. Examples of modified nucleotides are also described in U.S. Pat. Nos. 7,427,673, and 7,057,026, the disclosures of which are incorporated herein by reference in their entireties.

Additional exemplary SBS systems and methods which can be utilized with the methods and systems described herein are described in U.S. Patent Application Publication No. 2007/0166705, U.S. Patent Application Publication No. 2006/0188901, U.S. Pat. No. 7,057,026, U.S. Patent Application Publication No. 2006/0240439, U.S. Patent Application Publication No. 2006/0281109, PCT Publication No. WO 05/065814, U.S. Patent Application Publication No. 2005/0100900, PCT Publication No. WO 06/064199, PCT Publication No. WO 07/010,251, U.S. Patent Application Publication No. 2012/0270305 and U.S. Patent Application Publication No. 2013/0260372, the disclosures of which are incorporated herein by reference in their entireties.

Some embodiments can utilize detection of four different nucleotides using fewer than four different labels. For example, SBS can be performed utilizing methods and systems described in the incorporated materials of U.S. Patent Application Publication No. 2013/0079232. As a first example, a pair of nucleotide types can be detected at the same wavelength, but distinguished based on a difference in intensity for one member of the pair compared to the other, or based on a change to one member of the pair (e.g. via chemical modification, photochemical modification or physical modification) that causes apparent signal to appear or disappear compared to the signal detected for the other member of the pair. As a second example, three of four different nucleotide types can be detected under particular conditions while a fourth nucleotide type lacks a label that is detectable under those conditions, or is minimally detected under those conditions (e.g., minimal detection due to background fluorescence, etc.). Incorporation of the first three nucleotide types into a nucleic acid can be determined based on presence of their respective signals and incorporation of the fourth nucleotide type into the nucleic acid can be determined based on absence or minimal detection of any signal. As a third example, one nucleotide type can include label(s) that are detected in two different channels, whereas other nucleotide types are detected in no more than one of the channels. The aforementioned three exemplary configurations are not considered mutually exclusive and can be used in various combinations. An exemplary embodiment that combines all three examples, is a fluorescent-based SBS method that uses a first nucleotide type that is detected in a first channel (e.g. dATP having a label that is detected in the first channel when excited by a first excitation wavelength), a second nucleotide type that is detected in a second channel (e.g. dCTP having a label that is detected in the second channel when excited by a second excitation wavelength), a third nucleotide type that is detected in both the first and the second channel (e.g. dTTP having at least one label that is detected in both channels when excited by the first and/or second excitation wavelength) and a fourth nucleotide type that lacks a label that is not, or minimally, detected in either channel (e.g. dGTP having no label).

Another exemplary embodiment, is a fluorescent-based SBS method that uses a first nucleotide type that is detected in a first channel (e.g. dATP having a label that is detected in the first channel when excited by a first excitation wavelength), a second nucleotide type that is detected in a second channel (e.g. dCTP having a label that is detected in the second channel when excited by a second excitation wavelength), a third nucleotide type that is detected in both the first and the second channel (e.g. dTTP having at least one label that is detected in both channels when excited by the first and/or second excitation wavelength), a fourth nucleotide type that lacks a label that is not, or minimally, detected in either channel (e.g. dGTP having no label) and a fifth nucleotide type that is detected in the second channel when excited by a first excitation wavelength (e.g. dPaTP having a label that is excited by the first excitation wavelength, but that emits in the second channel).

Another exemplary embodiment, is a fluorescent-based method that uses four channels, wherein a first nucleotide type emits in channel 1 (e.g. dATP), a second nucleotide type emits in channel 2 (e.g. dTTP), a third nucleotide type emits in channel 3 (e.g. dCTP), a fourth nucleotide type emits in channel 4 (e.g. dGTP) and a fifth nucleotide does not emit in channels 1 through 4 (e.g. dPaTP), it may contain no flour or it may contain a flour that emits in a fifth channel. For example, the non-natural/unnatural base may be detected using a dye set with an orthogonal excitation/emission characteristic, such as, but not limited to, a FRET dye (see Table 2).

Any combination of detection methods may be used to identify the four natural bases and the fifth non-natural/unnatural base.

Further, as described in the incorporated materials of U.S. Patent Application Publication No. 2013/0079232, sequencing data can be obtained using a single channel. In such so-called one-dye sequencing approaches, the first nucleotide type is labeled but the label is removed after the first image is generated, and the second nucleotide type is labeled only after a first image is generated. The third nucleotide type retains its label in both the first and second images, and the fourth nucleotide type remains unlabeled in both images.

Some embodiments can utilize sequencing by ligation techniques. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. As with other SBS methods, images can be obtained following treatment of an array of nucleic acid features with the labeled sequencing reagents. Each image will show nucleic acid features that have incorporated labels of a particular type. Different features will be present or absent in the different images due the different sequence content of each feature, but the relative position of the features will remain unchanged in the images. Images obtained from ligation-based sequencing methods can be stored, processed and analyzed. Exemplary SBS systems and methods which can be utilized with the methods and systems described herein are described in U.S. Pat. Nos. 6,969,488, 6,172,218, and 6,306,597, the disclosures of which are incorporated herein by reference in their entireties.

Some embodiments can utilize nanopore sequencing (Deamer, D. W. & Akeson, M. “Nanopores and nucleic acids: prospects for ultrarapid sequencing.” Trends Biotechnol. 18, 147-151 (2000); Deamer, D. and D. Branton, “Characterization of nucleic acids by nanopore analysis”. Acc. Chem. Res. 35:817-825 (2002); Li, J., M. Gershow, D. Stein, E. Brandin, and J. A. Golovchenko, “DNA molecules and configurations in a solid-state nanopore microscope” Nat. Mater. 2:611-615 (2003), the disclosures of which are incorporated herein by reference in their entireties). In such embodiments, the target nucleic acid passes through a nanopore. The nanopore can be a synthetic pore or biological membrane protein, such as a-hemolysin. As the target nucleic acid passes through the nanopore, each base-pair can be identified by measuring fluctuations in the electrical conductance of the pore. (U.S. Pat. No. 7,001,792; Soni, G. V. & Meller, “A. Progress toward ultrafast DNA sequencing using solid-state nanopores.” Clin. Chem. 53, 1996-2001 (2007); Healy, K. “Nanopore-based single-molecule DNA analysis.” Nanomed. 2, 459-481 (2007); Cockroft, S. L., Chu, J., Amorin, M. & Ghadiri, M. R. “A single-molecule nanopore device detects DNA polymerase activity with single-nucleotide resolution.” J. Am. Chem. Soc. 130, 818-820 (2008), the disclosures of which are incorporated herein by reference in their entireties). Data obtained from nanopore sequencing can be stored, processed and analyzed. In particular, the data can be treated as an image in accordance with the exemplary treatment of optical images and other images.

Some embodiments can utilize methods involving the real-time monitoring of DNA polymerase activity. Nucleotide incorporations can be detected through fluorescence resonance energy transfer (FRET) interactions between a fluorophore-bearing polymerase and y-phosphate-labeled nucleotides as described, for example, in U.S. Pat. Nos. 7,329,492 and 7,211,414 (each of which is incorporated herein by reference) or nucleotide incorporations can be detected with zero-mode waveguides as described, for example, in U.S. Pat. No. 7,315,019 (which is incorporated herein by reference) and using fluorescent nucleotide analogs and engineered polymerases as described, for example, in U.S. Pat. No. 7,405,281 and U.S. Patent Application Publication No. 2008/0108082 (each of which is incorporated herein by reference). The illumination can be restricted to a zeptoliter-scale volume around a surface-tethered polymerase such that incorporation of fluorescently labeled nucleotides can be observed with low background (Levene, M. J. et al. “Zero-mode waveguides for single-molecule analysis at high concentrations.” Science 299, 682-686 (2003); Lundquist, P. M. et al. “Parallel confocal detection of single molecules in real time.” Opt. Lett. 33, 1026-1028 (2008); Korlach, J. et al. “Selective aluminum passivation for targeted immobilization of single DNA polymerase molecules in zero-mode waveguide nano structures.” Proc. Natl. Acad. Sci. USA 105, 1176-1181 (2008), the disclosures of which are incorporated herein by reference in their entireties). Images obtained from such methods can be stored, processed and analyzed.

Some SBS embodiments include detection of a proton released upon incorporation of a nucleotide into an extension product. For example, sequencing based on detection of released protons can use an electrical detector and associated techniques that are commercially available from Ion Torrent (Guilford, CT, a Life Technologies subsidiary) or sequencing methods and systems described in US 2009/0026082 A1; US 2009/0127589 A1; US 2010/0137143 A1; or US 2010/0282617 A1, each of which is incorporated herein by reference. Methods set forth herein for amplifying target nucleic acids using kinetic exclusion can be readily applied to substrates used for detecting protons. More specifically, methods set forth herein can be used to produce clonal populations of amplicons that are used to detect protons.

The above SBS methods can be advantageously carried out in multiplex formats such that multiple different target nucleic acids are manipulated simultaneously. In particular embodiments, different target nucleic acids can be treated in a common reaction vessel or on a surface of a particular substrate. This allows convenient delivery of sequencing reagents, removal of unreacted reagents and detection of incorporation events in a multiplex manner. In embodiments using surface-bound target nucleic acids, the target nucleic acids can be in an array format. In an array format, the target nucleic acids can be typically bound to a surface in a spatially distinguishable manner. The target nucleic acids can be bound by direct covalent attachment, attachment to a bead or other particle or binding to a polymerase or other molecule that is attached to the surface. The array can include a single copy of a target nucleic acid at each site (also referred to as a feature) or multiple copies having the same sequence can be present at each site or feature. Multiple copies can be produced by amplification methods such as, bridge amplification or emulsion PCR as described in further detail below.

The methods set forth herein can use arrays having features at any of a variety of densities including, for example, at least about 10 features/cm2, 100 features/cm2, 500 features/cm2, 1,000 features/cm2, 5,000 features/cm2, 10,000 features/cm2, 50,000 features/cm2, 100,000 features/cm2, 1,000,000 features/cm2, 5,000,000 features/cm2, or higher.

In an embodiment, the non-natural/unnatural base may be identified in combination with the four natural bases. For example, identification of incorporation of five bases and/or five distinguishable signals, including, but not limited to, identification of a signal identified by the absence of a signal. For example, the four natural nucleotides may be labeled with an identifiable and distinguishable marker and the non-natural/unnatural base identified by the absence of an actual signal. As will now be recognized, any of the five bases may lack the signal, so long as the remaining four bases can be identified and distinguished from one another and the absence of a signal. In particular embodiments, this method of identifying five bases is used on repaired target polynucleotides comprising a high fidelity non-natural/unnatural base; with the distinguishable signals A, T, G, C, and a high fidelity non-natural/unnatural base.

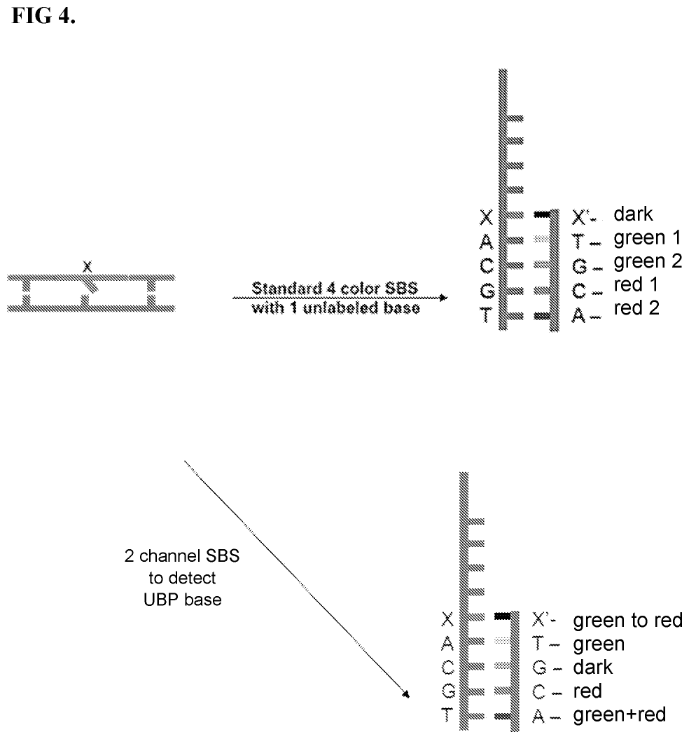

Identification of the non-natural/unnatural base can be done using 2-channel detection, as shown in FIG. 4, Tables 1 and 2, or 4-channel detection.

TABLE 1
Detection of five bases by extending 2-channel chemistry.

| | Green Excitation | | Red Excitation |
|---|---|---|---|
| | Green Emission | Red Emission | Red Emission |
| A | X | ◯ | X |
| G | ◯ | ◯ | ◯ |
| C | ◯ | ◯ | X |
| T | X | ◯ | ◯ |
| X | ◯ | X | ◯ |

Where “X” indicates a signal and “◯” indicates the absence of a signal.

Detection of five bases by extending 2-channel chemistry.

| | Green 1 Emission | Red Emission | Green 2 Emission |
|---|---|---|---|
| A | X | X | ◯ |
| G | ◯ | ◯ | ◯ |
| C | ◯ | X | ◯ |
| T | X | ◯ | ◯ |
| X | ◯ | ◯ | X |

Where “X” indicates a signal and “◯” indicates the absence of a signal and Green 1 is distinguishable from Green 2.

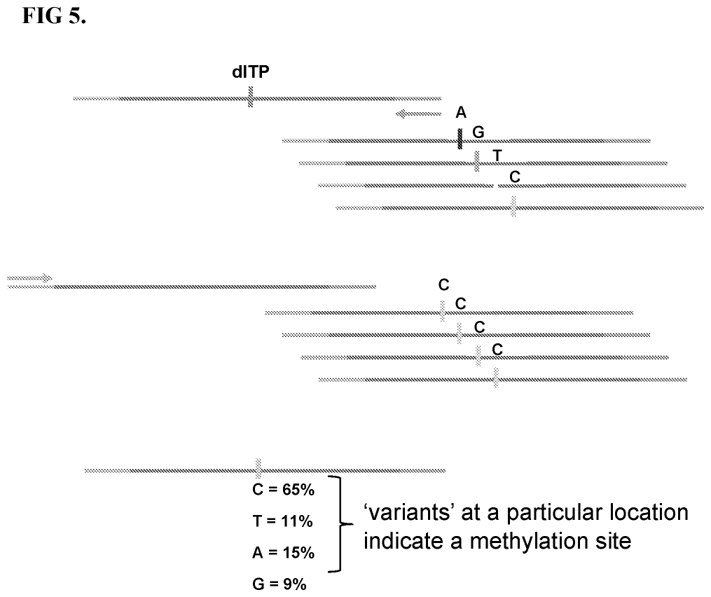

In an embodiment, a low fidelity non-natural/unnatural base may be identified in combination with the four natural bases. As depicted in FIG. 5, the amplification of a strand containing a low fidelity non-natural/unnatural base will lead to the incorporation of one the natural bases in the daughter strand. However, when the complementary strand is amplified, it will always incorporate a C at that location. After sequencing is complete, the sites with variants a particular location are identified as being methylated cytosine. In addition, to aid in the alignment of locations with variants for a particular location, the double stranded nucleic acid may be fragmented and labeled 3′ and or 5′ with Unique Molecular Identifiers (UMIs) as is well known the art prior the treatment of the double stranded nucleic acid with the glycosylase. Unique molecular indices or unique molecular identifiers (UMIs) are sequences of nucleotides applied to or identified in DNA molecules that may be used to distinguish individual DNA molecules from one another. Since UMIs are used to identify DNA molecules, they are also referred to as unique molecular identifiers. See, e.g., Kivioja, Nature Methods 9, 72-74 (2012). UMIs may be sequenced along with the DNA molecules with which they are associated to determine whether the read sequences are those of one source DNA molecule or another. The term “UMI” is used herein to refer to both the sequence information of a polynucleotide and the physical polynucleotide per se.

Commonly, multiple instances of a single source molecule are sequenced. In the case of sequencing by synthesis using Illumina's sequencing technology, the source molecule may be PCR amplified before delivery to a flow cell.

UMIs are similar to bar codes, which are commonly used to distinguish reads of one sample from reads of other samples, but UMIs are instead used to distinguish one source DNA molecule from another when many DNA molecules are sequenced together. Because there may be many more DNA molecules in a sample than samples in a sequencing run, there are typically many more distinct UMIs than distinct barcodes in a sequencing run.

As mentioned, UMIs may be applied to or identified in individual DNA molecules. In some implementations, the UMIs may be applied to the DNA molecules by methods that physically link or bond the UMIs to the DNA molecules, e.g., by ligation or transposition through polymerase, endonuclease, transposases, etc. These “applied” UMIs are therefore also referred to as physical UMIs. In some contexts, they may also be referred to as exogenous UMIs. The UMIs identified within source DNA molecules are referred to as virtual UMIs. In some context, virtual UMIs may also be referred to as endogenous UMI.

Physical UMIs may be defined in many ways. For example, they may be random, pseudo-random or partially random, or nonrandom nucleotide sequences that are inserted in adapters or otherwise incorporated in source DNA molecules to be sequenced. In some implementations, the physical UMIs may be so unique that each of them is expected to uniquely identify any given source DNA molecule present in a sample. The collection of adapters is generated, each having a physical UMI, and those adapters are attached to fragments or other source DNA molecules to be sequenced, and the individual sequenced molecules each has a UMI that helps distinguish it from all other fragments. In such implementations, a very large number of different physical UMIs (e.g., many thousands to millions) may be used to uniquely identify DNA fragments in a sample.

Of course, the physical UMI must have a sufficient length to ensure this uniqueness for each and every source DNA molecule. In some implementations, a less unique molecular identifier can be used in conjunction with other identification techniques to ensure that each source DNA molecule is uniquely identified during the sequencing process. In such implementations, multiple fragments or adapters may have the same physical UMI. Other information such as alignment location or virtual UMIs may be combined with the physical UMI to uniquely identify reads as being derived from a single source DNA molecule/fragment. In some implementations, adaptors include physical UMIs limited to a relatively small number of nonrandom sequences, e.g., 120 nonrandom sequences. Such physical UMIs are also referred to as nonrandom UMIs. In some implementations, the nonrandom UMIs may be combined with sequence position information, sequence position, and/or virtual UMIs to identify reads attributable to a same source DNA molecule. The identified reads may be combined to obtain a consensus sequence that reflects the sequence of the source DNA molecule as described herein. Using physical UMIs, virtual UMIs, and/or alignment locations, one can identify reads having the same or related UMIs or locations, which identified reads can then be combined to obtain one or more consensus sequences. The process for combining reads to obtain a consensus sequence is also referred to as “collapsing” reads.

In an exemplary embodiment, the non-natural/unnatural base read out may be marked as a cytosine for the purpose of mapping to a reference genome. The non-natural/unnatural base read out may be marked as a 5-meC or 5-hmeC, before or after mapping to a reference genome, and then analyzed to identify and/or visualize the methylome.

In another exemplary embodiment, the present invention provides a kit for detecting methylated cytosine in a target DNA. The kit may include one or more of the following: an enzyme having DNA glycosylase activity that selectively removes methylated cytosine so as to create an abasic site, a DNA AP lyase and/or AP endonuclease and at least one non-natural base capable of repairing the abasic site. In another exemplary embodiment, the present invention provides a kit for detecting methylated cytosine in a target DNA. The kit may include one or more of the following: an enzyme having DNA glycosylase activity that selectively removes methylated cytosine so as to create an abasic site, a DNA AP lyase and/or AP endonuclease and two non-natural bases with at least one being capable of repairing the n site and the second being having high fidelity during incorporation in the repaired target DNA.

What is claimed is:

1. A method for detecting methylated cytosine in a target DNA, the method comprising:
treating the target DNA with an enzyme having DNA glycosylase activity that selectively removes methylated cytosine so as to create an abasic site;
breaking the phosphate backbone of the target DNA at the abasic site with a DNA AP lyase or AP endonuclease;
repairing the abasic site by inserting a non-natural base into the abasic site to generate repaired target DNA; and
sequencing the repaired target DNA so as to identify positions in the repaired target DNA that contain the non-natural base thereby detecting methylated cytosine in the target DNA.

2. The method according to claim 1, wherein repairing the abasic site comprises treating the abasic site with an endonuclease IV and a 3′ phosphatase.

3. The method according to claim 1, wherein repairing the abasic site comprises treating the abasic site with a 3′ phosphatase.

4. The method according to claim 1, wherein the DNA glycosylase has EC 3.2.2.-activity.

5. The method according to claim 1, wherein the DNA AP lyase has EC 4.2.99.18 activity.

6. The method according to claim 2, wherein the Endonuclease IV has EC 3.1.21.2 activity.

7. The method according to claim 2, wherein the Endonuclease IV has EC 3.1.21.9 activity.

8. The method of claim 1, wherein repairing the abasic site comprises treating the abasic site with a polymerase having EC 2.7.7.7 activity.

9. The method of claim 1, wherein the enzyme that selectively removes the methylated nucleotide comprises DNA glycosylase activity and abasic site lyase activity.

10. The method according to claim 1, wherein repairing the abasic site further comprises treating the abasic site with a ligase.

11. The method according to claim 10, wherein the DNA Ligase has EC 6.5.1 activity.

12. The method according to claim 11, wherein the DNA Ligase has EC 6.5.1.1 or 6.5.1.2 activity.

13. The method according to claim 2, wherein the 3′ phosphatase has EC 3.1.3.32 activity.

14. The method according to claim 1, wherein the non-natural base is a base that pairs with low fidelity to multiple nucleotides.

15. The method according to claim 14, wherein identifying positions in the repaired target DNA that contain the non-natural base comprises identifying nucleotide sites in the repaired target DNA having statistically significant nucleotide infidelity.

16. The method according to claim 14, wherein the non-natural nucleotide is a universal base.

17. The method of claim 16, wherein the universal base is selected from the group consisting of deoxyInosine and 5-Nitroindole.

18. The method of claim 16, the repaired target DNA comprises a unique molecular identifier (UMI) sequence.

19. The method according to claim 1, further comprising pairing a second non-natural nucleotide with high fidelity to the non-natural base.

20. The method according to claim 1, wherein sequencing the repaired target DNA comprises amplifying the repaired target DNA.

21. A kit for detecting methylated cytosine in a target DNA, the kit comprising:
a) an enzyme having DNA glycosylase activity that selectively removes methylated cytosine so as to create an abasic site;
b) a DNA AP lyase or AP endonuclease which is capable of breaking the phosphate backbone of the target DNA at the abasic site; and
c) at least one a non-natural base capable of repairing the abasic site by inserting into the abasic site to generate repaired target DNA.

22. The kit of claim 18, further comprising a second non-natural nucleotide that pairs with high fidelity to the first non-natural nucleotide.

23. The kit of claim 19, further comprising a polymerase having greater than 50% fidelity during incorporation of the second non-natural nucleotide at the repaired abasic site(s) in the repaired target DNA.

24. The method according to claim 4, wherein the enzyme that selectively removes the methylated nucleotide is selected from the group consisting of ROS1, DEMETER (DMA), DME Like (DML) 2 and DME Like (DML) 3.
